(19) United States
(12) Patent Application Publication
(10) Pub. No.: US 2017/0357753 A1
(43) Pub. Date: Dec. 14, 2017

(54) DIRECT ESTIMATION OF PATIENT ATTRIBUTES BASED ON MRI BRAIN ATLASES

(71) Applicant: THE JOHNS HOPKINS UNIVERSITY, Baltimore, MD (US)

(72) Inventors: Susumu Mori, Ellicott City, MD (US); Michael I. Miller, Baltimore, MD (US); Dan Wu, Baltimore, MD (US)

(21) Appl. No.: 15/602,578

(22) Filed: May 23, 2017

Related U.S. Application Data
(60) Provisional application No. 62/340,023, filed on May 23, 2016.

(51) Int. Cl. G06F 19/00; A61B 5/00; G06F 17/30

(52) U.S. Cl. CPC G06F 19/321; G06F 17/30572; G06F 19/345; G06F 19/324; A61B 5/4088

(57) ABSTRACT

The present invention is directed to a context-based image retrieval (CBIR) system for disease estimation based on the multi-atlas framework, in which the demographic and diagnostic information of multiple atlases are weighted and fused to generate an estimated diagnosis, on a structure-by-structure basis. The present invention demonstrates high accuracy in age estimation, as well as diagnostic estimation in Alzheimer's disease. The system and the pathology-based multi atlases can be used to estimate various types of disease and pathology with the choice of patient attributes. The present invention is also directed to a method of context-based image retrieval.

### CROSS REFERENCE TO RELATED APPLICATION

This application claims the benefit of U.S. Provisional Patent Application No. 62/340,023 filed on May 23, 2016, which is incorporated by reference, herein, in its entirety.

### GOVERNMENT SUPPORT

The present invention was made with government support under EB015909, EB17638, and NS084957 awarded by the National Institutes of Health. The government has certain rights in the present invention.

### FIELD OF THE INVENTION

The present invention relates generally to medical imaging. More particularly, the present invention relates to a method for direct estimation of patient attributes based on MRI brain atlases.

### BACKGROUND OF THE INVENTION

Anatomical MRI is an indispensable tool to diagnose various brain diseases. Three types of MRI methods, T1-weighted, T2-weighted, and FLAIR, have been most widely used clinically. Based on specific features that appear in these images, radiologists estimate the likely causes of the features and arrive at the best medical judgment. There are three types of critical information radiologists extract from the images: the type, degree, and location of the features. These features are then compared to their knowledge about the range of normal appearance at a given age of the patient. If considered abnormal, the type, degree, and location of the abnormality are documented in a radiological report. Radiologists often go one step further by performing a similarity search within their knowledge of various diseases and provide potential diagnoses. In the field of computer vision, this is a type of context-based image retrieval (CBIR). Namely, there is a knowledge database that contains images and associated text-based attributes, such as demographic, clinical, and diagnostic information. When an image of a new patient is provided, along with his/her demographic and clinical information, past cases with similar features are extracted, together with the desired diagnostic information.

The degree of abnormality varies widely among different brain diseases. Ischemic infarction and tumor are diseases that often demonstrate large effect sizes, and MRI is considered one of the most effective diagnostic tools. At the other end of the spectrum are psychiatric diseases, for which MRI is not considered effective enough in routine clinical diagnosis.

Dementia populations are located in the middle of the spectrum. Various dementia diseases with different causes and time courses are known to demonstrate brain atrophy in specific brain structures. However, this is compounded by the natural course of brain atrophy in aging brains, ambiguous correlations between the amount of the atrophy and clinical performance, and mediocre specificity between brain atrophy features and specific causes of the dementia. Through past clinical experience and research, loose relationships between brain pathology and anatomical features have been established. For example, hippocampal atrophy is believed to be a hallmark of Alzheimer's disease, and frontotemporal dementia usually accompanies atrophy of the frontal and temporal lobes. However, such correlations are not strong enough for use of these anatomical features alone for diagnosis. As a result, MRI has been used only as secondary information for the diagnosis of dementia.

The above discussion indicates that MRI data are only a weakly discriminating factor to differentiate certain brain pathologies. For dementia populations, all available clinical data are only weakly discriminating factors, which is the primary cause of the challenge clinicians are facing in patient care. In this situation, it is important to quantitatively analyze each clinical modality, combine the results across modalities, and provide the meaning of certain observed features in statistical terms. For image analysis, the classic approach is to homogenize the patient population into a specific dementia group based on clinical symptoms (e.g., MCI, AD, etc.) and to perform voxel-based analysis to identify certain anatomical features that differentiate the population from a control group. This approach, however, is compounded by the fact that the “homogenized” population still has a substantial amount of variability in the nature, degree, and location of the abnormalities and, thus, population-averaging of the location information (voxels) does not necessarily increase the statistical power. This is because there are no strongly discriminating factors that would purify the population to a single pathological state and also because aged populations usually contain multiple pathologies. Namely, a heterogeneous “nature” and “degree” of pathologies could exist in different “locations.” The present invention uses a CBIR approach, and extracts diagnostic information from a knowledge database that consisted of a heterogeneous dementia population through image-feature matching.

In the past, CBIR has been attempted for several radiological images, such as lung CT and mammography. For brain MRI, machine-learning approaches, such as support vector machine based on the voxel intensities in the entire brain, or image similarity search based on voxel-based mutual information, or segmentation-based feature matching have been tested. What is common to these past studies is that non-image patient attributes (such as potential diagnosis) were obtained based on the anatomical features of the entire brain.

Accordingly, there is a need in the art for a method for direct estimation of patient attributes based on MRI brain atlases.

### SUMMARY OF THE INVENTION

The foregoing needs are met, to a great extent, by the present invention which provides a method for estimation of patient attributes including providing a database framework of multiple brain atlases. The method includes weighing demographic and diagnostic information of the multiple brain atlases. The method includes fusing the demographic and diagnostic information based on the weighing of the multiple brain atlases. The method further includes generating an estimated diagnosis on a structure by structure basis.

In accordance with an aspect of the present invention, the method includes using a database framework based on magnetic resonance (MR) images. The method includes using a context-based image retrieval system. The method includes estimating various types of disease and pathology with the choice of patient attributes. The method includes diagnostic estimation in Alzheimer's disease. The method includes building the multiple brain atlases with images from healthy volunteers with a wide range of age and pathological states. The method includes performing multiple-atlas segmentation based on label-by-label atlas weighting. The method includes using atlases containing a number of anatomical structures, wherein each structure has associated information for age, diagnosis, and interesting atlas properties. The method also includes building aging and diagnosis probability maps for each of the number of anatomical structures. Additionally, the method includes generating and displaying maps associated with the number of anatomical structures and generating and displaying maps and visual representations of data associated with method.

In accordance with another aspect of the present invention, a system for estimation of patient attributes includes a database framework of multiple brain atlases. The system also includes a non-transitory computer readable medium programmed for weighing demographic and diagnostic information of the multiple brain atlases. The non-transitory computer readable medium is also programmed for fusing the demographic and diagnostic information based on the weighing of the multiple brain atlases and generating an estimated diagnosis on a structure by structure basis.

In accordance with yet another aspect of the present invention, the system includes using a database framework based on magnetic resonance (MR) images. The system includes using a context-based image retrieval system. The system can be used for diagnostic estimation in Alzheimer's disease. The system includes performing multiple-atlas segmentation based on label-by-label atlas weighting. The system also includes using atlases containing a number of anatomical structures, wherein each structure has associated information for age, diagnosis, and interesting atlas properties and building aging and diagnosis probability maps for each of the number of anatomical structures. Additionally, the system includes generating and displaying maps associated with the number of anatomical structures and generating and displaying maps and visual representations of data associated with method.

### BRIEF DESCRIPTION OF THE DRAWING

The accompanying drawings provide visual representations, which will be used to more fully describe the representative embodiments disclosed herein and can be used by those skilled in the art to better understand them and their inherent advantages. In these drawings, like reference numerals identify corresponding elements and:

FIG. 1 illustrates a schematic diagram showing the concepts of context-based imaging retrieval (CBIR) based analysis and conventional region-of-interest (ROI) based analysis.

FIGS. 2A and 2B illustrate graphical views of data according to an embodiment of the present invention.

FIGS. 3A and 3B illustrate image views of whole-brain mapping of the R² and linear correlation coefficients of the linear regression between the estimated age and actual age in each structure, overlaid on a T1-weighted image.

FIG. 4 illustrates graphical views of R² of the linear regression between the structural volume and age (dark grey bar), compared to the R² of the linear regression between the CBIR-based estimation and age, in 289 structures over the whole brain.

FIGS. 5A and 5B illustrate graphical views of dementia probabilities and control/MCI/AD probabilities.

FIG. 6 illustrates whole-brain mapping of the estimated ADAS.11 scores in the normal elderly, MCI, and AD test subjects.

FIGS. 7A and 7B illustrate graphical views of linear regressions, according to an embodiment of the present invention.

FIGS. 8A and 8B illustrate image views of whole brain mapping of the R2, according to an embodiment of the present invention.

### DETAILED DESCRIPTION

The presently disclosed subject matter now will be described more fully hereinafter with reference to the accompanying Drawings, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

The present invention is directed to a context-based image retrieval (CBIR) system for disease estimation based on the multi-atlas framework, in which the demographic and diagnostic information of multiple atlases are weighted and fused to generate an estimated diagnosis, on a structure-by-structure basis. The present invention demonstrates high accuracy in age estimation, as well as diagnostic estimation in Alzheimer's disease. The system and the pathology-based multi atlases can be used to estimate various types of disease and pathology with the choice of patient attributes. The present invention is also directed to a method of context-based image retrieval.

The present invention is directed to a unique approach to location-dependent feature analysis, using a multiple-atlas brain segmentation paradigm framework. In the atlas-based segmentation approach there is at least one atlas with pre-defined structures, which is warped to a patient image, thus transferring the structural definition for automated segmentation. In the multiple-atlas approach there are multiple, typically more than ten, atlases which are all warped to a patient image. This leads to ten different results, for example, of the hippocampus boundaries, followed by an arbitration process to derive the best estimation of the structure. During the arbitration, if all ten atlases receive equal weighting, majority voting prevails. In more advanced approaches, each atlas receives weighting based on anatomical similarity measures, such as the voxel intensity. In the Bayes approach, the conditional probability of a segmentation label is determined by the likelihood of the image at that location as a function of the label value. By using multiple atlases and weighting among them, atlases with similar anatomy and better registration accuracy can be chosen, and thereby, more accurate structural boundary definitions. Depending on algorithms, this operation is performed in a voxel-by-voxel or label-by-label manner.

The content of the atlas library is often the subject of various interesting questions. These include how many atlases are needed, whether they should be age-matched, or whether they should include pathological cases. If an 80-year-old AD patient image is presented and if all the atlases are from healthy subjects, none of the atlases may have a similar degree of brain atrophy and the registration accuracy could be poor. The present invention includes prepared atlases that contain images from healthy volunteers with a wide range of age and pathological states, including patients with mild cognitive impairment (MCI), and Alzheimer's disease (AD). Then, multiple-atlas segmentation was performed based on label-by-label atlas weighting. Instead of focusing on the degree of segmentation accuracy, the present invention focuses on atlas weighting as a measure of diagnostic voting from multiple atlases. This is natural because the solution to the Bayes problem of disease decision-making views structural definitions as hidden variables for which the conditional probability of the disease-type conditioned on the image integrates over. This implies the optimum Bayes decision rule would only estimate ancillary variables such as segmentation labels as a convenience, for example if they were to form completion variables to make an optimization procedure such as the EM algorithm work. Atlases associated with the present invention contain 289 anatomical structures, and for each structure, interesting atlas properties, their age, and diagnosis are measured. This leads to aging and diagnosis probability maps for each structure. These maps can be generated and displayed by a computing device associated with the present invention. Any other maps or visual representations of the data associated with the present invention can also be displayed. A part of the atlas populations were used as test data to determine whether the tool could accurately estimate the age and diagnosis of the test data.

In multi-atlas based segmentation, the parcellation profiles of the target image from each atlas, after registration, are combined according to certain atlas weighting and fusion schemes. The registration of the present invention is achieved first by affine transformation, and then iterative Large Deformation Diffeomorphic Metric Mapping (LDDMM), along with iterative inhomogeneity corrections. Let IT be the target image, IAi (i=1, 2, . . . , N) be the atlas images after warping to the target image, and LA i be the label images associated with the warped atlases. A weighted voting approach was used for label fusion:

\hat{p}(l|x,I_{T})=Σᵢ₌₁^{N}w_{A}ⁱ(x)·p(l|x,I_{A}ⁱ) Equation 1

where \hat{p}(l|x, I_{T}) is the estimated probability of voxel x being labeled l in the target image, and l=1, 2, . . . , L with L the total number of labels. (l|x, I_{A}ⁱ) is the probability of voxel x being labeled as l in the warped atlas, with (l|x, I_{A}ⁱ)=1 when L_{A}ⁱ(x)=l and p(l|x, I_{A}ⁱ)=0 otherwise. w_{A}ⁱ(x) represents the atlas weighting term that measures the similarity between the target and atlas i at voxel x, with Σᵢ₌₁^{N}=w_{A}ⁱ(x)=1. The atlas weighting used in the present invention is described further herein. The final segmentation can be obtained by the Bayes maximum a posteriori (MAP) estimation,

$${L}_{T}^{i} ( x ) = {argmax}_{l ∈ \{ 1 , … , L \}} \hat{p} ( l \vert x , {I}_{T} ) .$$

Atlas-weighting is essential in the weighted multi-atlas voting, as well as a key factor in the CBIR-based disease estimation. Atlas-weightings are assigned to each individual structure, based on the intensity similarity on a label-by-label basis, as opposed to a voxel-by-voxel based approach. The similarity is measured based on the local intensity match along the boundary of each structural label between the target and the warped atlases. The boundary voxels are chosen rather than all voxels in the label, assuming the image intensities inside the structure relatively are homogeneous and the boundary voxels are more sensitive to the structural similarity. Let Nₓ=[x₁, x₂, . . . , x_{K}] be a vector of voxels in a local neighborhood patch of radius r×r×r centered on a boundary voxel x, then the similarity measure s_{A}ⁱ(x) of a warped atlas i is computed b

s_{A}ⁱ(x)=corr(I_{A}ⁱ(Nₓ),I_{T}(Nₓ)) Equation 2

where corr(•) is the Pearson correlation coefficient

$$corr ( {I}_{A}^{i} ( {N}_{x} ) , {I}_{T} ( {N}_{x} ) ) = \frac{E [ ( {I}_{A}^{i} ( {N}_{x} ) - μ ( {I}_{A}^{i} ( {N}_{x} ) ) ) ( {I}_{T} ( {N}_{x} ) - μ ( {I}_{T} ( {N}_{x} ) ) ) ]}{σ ( {I}_{A}^{i} ( {N}_{x} ) ) σ ( {I}_{T} ( {N}_{x} ) )}$$

with E, μ, and σ being the expectation, mean, and standard deviation operations, respectively.

Because the warped atlases are already transformed through a deformation space to match the target image, in order to trace the features of the un-deformed atlases that reflect true anatomy of the disease population, a deformation cost is included in the atlas weighting. The deformation cost is calculated based on the deformation vector integrated over the deformation space

$$V \text{:} α ( {v}^{i} ) ∼ exp ( - \frac{1}{2} {‖ {v}^{i} ‖}_{V}^{2} )$$

Therefore, the atlas-weighting w_{A}ⁱ(l) of label l in atlas i, is a combined measure of the similarity and deformation cost integrated over the boundary voxels

w_{A}ⁱ(l)=Σ_{x∈b}_{_{A}}_{ⁱ}₍ₗ₎s_{A}ⁱ(x)·α(vⁱ(x)) Equation 3

where b_{A}ⁱ(l) denotes the boundary of label l in the warped atlas i.

The “context” used in CBIR here is atlas-weighting of the multiple atlases as introduced above. The multi-atlas data pool can cover various types of atlases, such pediatric, adult, and elderly atlases, as well as atlases from neurological diseases, such as Alzheimer's disease, Huntington's disease, and Parkinson's disease. Given the demographic and/or diagnostic information associated with each atlas, D(I_{A}ⁱ), the same information about the target image can be inferred by

D(I_{T}|l)=Σᵢ₌₁^{N}D(I_{A}ⁱ)·w_{A}ⁱ(l) Equation 4

where D(I_{T}|l) is the demographic or diagnosis of the target on a structure-by-structure basis.

The probability of the target image belonging to predefined diagnostic groups is calculated (e.g., normal/MCI/AD) by summing over the weightings of the atlases associated with that diagnostic group.

$$p ( {G}_{j} \vert {I}_{T} l ) = \frac{{\sum }_{i ∈ {G}_{j}} {w}_{A}^{i} ( l )}{{\sum }_{j = 1}^{J} {\sum }_{i ∈ {G}_{j}} {w}_{A}^{i} ( l )} \qquad Equation 5$$

where p(Gⱼ|I_{T}, l) is the probability of the target belonging to atlas group Gⱼ in terms of label l, with j=1, 2, . . . , J (the number of atlas groups).

The age specific multi-atlas dataset consisted of T1 atlases from pediatric population (4-12 yr, 20 atlases), midage population (20-50 yr, 20 atlases), and elderly (60-80 yr, 20 atlases). Another 10 atlases from each age group were used as test subjects. The age of the target can be estimated as a weighted sum of the ages of the atlas data according to Equation 4, where D(•) becomes an age measure.

The atlases are a subset of the MriCloud atlas repository (https://braingps.mricloud.org/atlasrepo), which were segmented to 289 structures with extensive manual correction. All images were acquired on Philips 3T, with image resolution in the range of 1.0×1.0×1.0 mm and 1.0×1.0×1.2 mm.

The dataset of the dementia specific multi-atlases consisted of MPRAGE images from the Alzheimer's Disease Neuroimaging Initiative (ADNI) (http://adni.loni.usc.edu/), with 20 atlases from the Alzheimer's disease (AD) population, 20 from the Mild Cognitive Impairment (MCI) population, and 20 from the normal elderly controls. Another 10 atlases from each group were used as test subjects (Table 1). All atlases are available at https://braingps.mricloud.org/atlasrepo. In addition to estimation of the disease categories (normal, MCI, and AD), patient attributes are estimated using one of widely used cognitive scores—the Alzheimer's Disease Assessment Scale-cognitive subscales with 11 items (ADAS.11). The ADAS.11 scores in the three groups are summarized in Table 1. The estimated ADAS.11 of the test data can be obtained according to Equation 4, where the D(•) will be the ADAS.11 measure. The probability of belonging to each disease category can be estimated based on Equation 5.

TABLE 1
ADNI data used for diagnosis estimation

| Group | No. | Usage | Age (years) | Diagnosis (ADAS.11) |
|---|---|---|---|---|
| Control | 20 | Atlas | 70.8 ± 8.3 | 4.53 ± 2.20 |
| Control | 10 | Test | 71.6 ± 2.5 | 6.57 ± 3.49 |
| MCI | 20 | Atlas | 73.1 ± 9.5 | 11.75 ± 2.81 |
| MCI | 10 | Test | 71.4 ± 8.7 | 12.78 ± 4.07 |
| AD | 20 | Atlas | 70.7 ± 11.0 | 17.05 ± 3.99 |
| AD | 10 | Test | 69.7 ± 12.3 | 20.67 ± 5.05 |

The ADNI data include data from Philips, SIEMENS, and GE at 1.5T and 3T. An even number of subjects from each protocol in each group (control, MCI, and AD) were used. The analysis, therefore, contains effects from image protocol differences. The inclusion of various MPRAGE protocols (all provided by the manufacturers) in the present invention is highly important to ensure that the observed biological effects will not be erased in practice when slightly different imaging parameters are used. However, it is also important to ensure that the observation is not due to differences in imaging parameters. The effect of scan protocol was evaluated by making the protocol type (6 types in ADNI) one of the covariates, and tested its significance in diagnosis estimation with two-way ANOVA and FDR correction. Statistical differences were found only in two structures out of the 289 brain segments (left fusiform gyrus and left subcortical white matter of the inferior temporal gyrus).

The ADNI was launched in 2003 by the National Institute on Aging (NIA), the National Institute of Biomedical Imaging and Bioengineering (NIBIB), the Food and Drug Administration (FDA), private pharmaceutical companies, and non-profit organizations, as a $60 million, five-year public/private partnership. The primary goal of ADNI has been to test whether serial magnetic resonance imaging (MRI), positron emission tomography (PET), other biological markers, and clinical and neuropsychological assessment can be combined to measure the progression of mild cognitive impairment (MCI) and early Alzheimer's disease (AD). Determination of sensitive and specific markers of very early AD progression is intended to aid researchers and clinicians in developing new treatments and monitoring the effectiveness of these treatments, as well as reducing the time and cost of clinical trials.

Once ages were estimated from the test data (n=30), they were compared with the actual patient age and correlation between the estimated and the actual ages was calculated by linear regression. Dementia estimation was similarly evaluated by linear regression between the estimated ADAS.11 scores and the actual ADAS.11 of the ADNI subjects (n=30). The R² was used to evaluate the good-ness-of-fit of the linear regression, and the p-value from the t-statistics was used to evaluate the significance of linear regression with False Discovery Rate (FDR) correction. To assess the significance of diagnosis estimation among ADNI groups, one-way analysis of variance (ANOVA) was used among the AD/MCI/normal test subjects (n=10 each), and the p-values from ANOVA tests were then corrected by FDR for multiple ROI comparisons. The ROI volumes were obtained from the segmentation to correlate with age or diagnosis, and compared the performances with the CBIR-based approach.

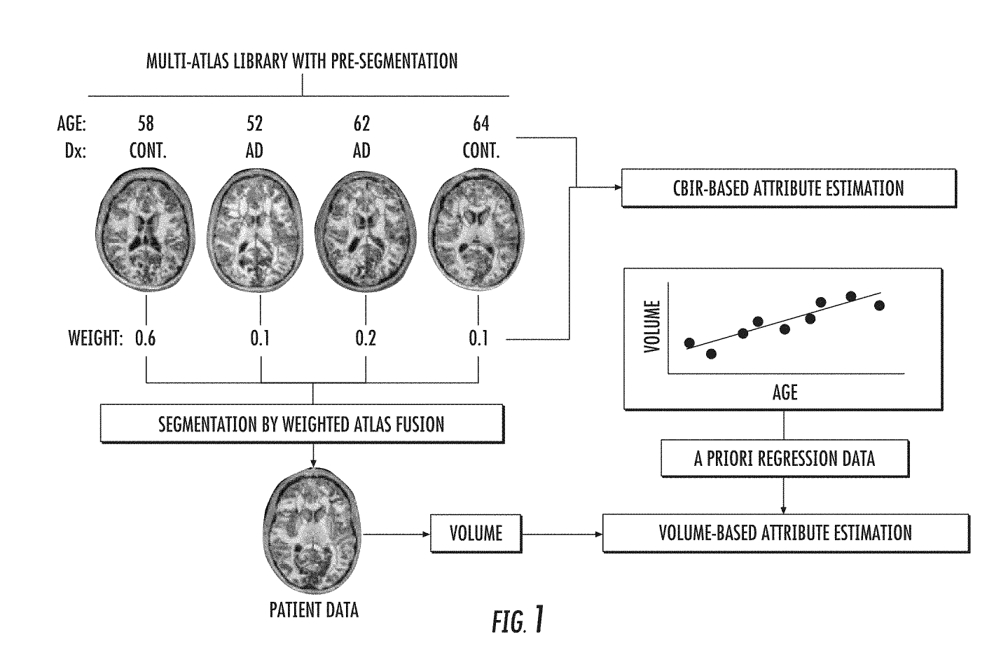

FIG. 1 illustrates a schematic diagram showing the concepts of context-based imaging retrieval (CBIR) based analysis and conventional region-of-interest (ROI) based analysis. In the CBIR approach, the similarity between patient image and the atlases were measured based on the image features, which is then used to weigh the diagnostic information associated with the multiple atlases to obtain a weighted estimation of the patient's attribute. In comparison, in ROI-based analysis, the multi-atlases are used to segment the image, and the volumes or intensities of the ROIs are used to estimate the patient's attribute, which relies on a priori regression data.

The two approaches are summarized in FIG. 1. The first approach is based on the CBIR, as described above. In this approach, the patient attributes (age and diagnosis) are obtained directly from the process of the multi-atlas pipeline and the resultant segmentation is merely a proof of procedural accuracy (as long as the segmentation is accurately performed, the segmentation results are discarded). The second approach is a more conventional method, in which the segmentation results (e.g., volumes) are compared with population-based regression for ages or diagnosis. The population-based regression needs to be established beforehand. The data is taken from the multi-atlas library to obtain the regression (volume vs age and volume vs diagnosis) for each ROI.

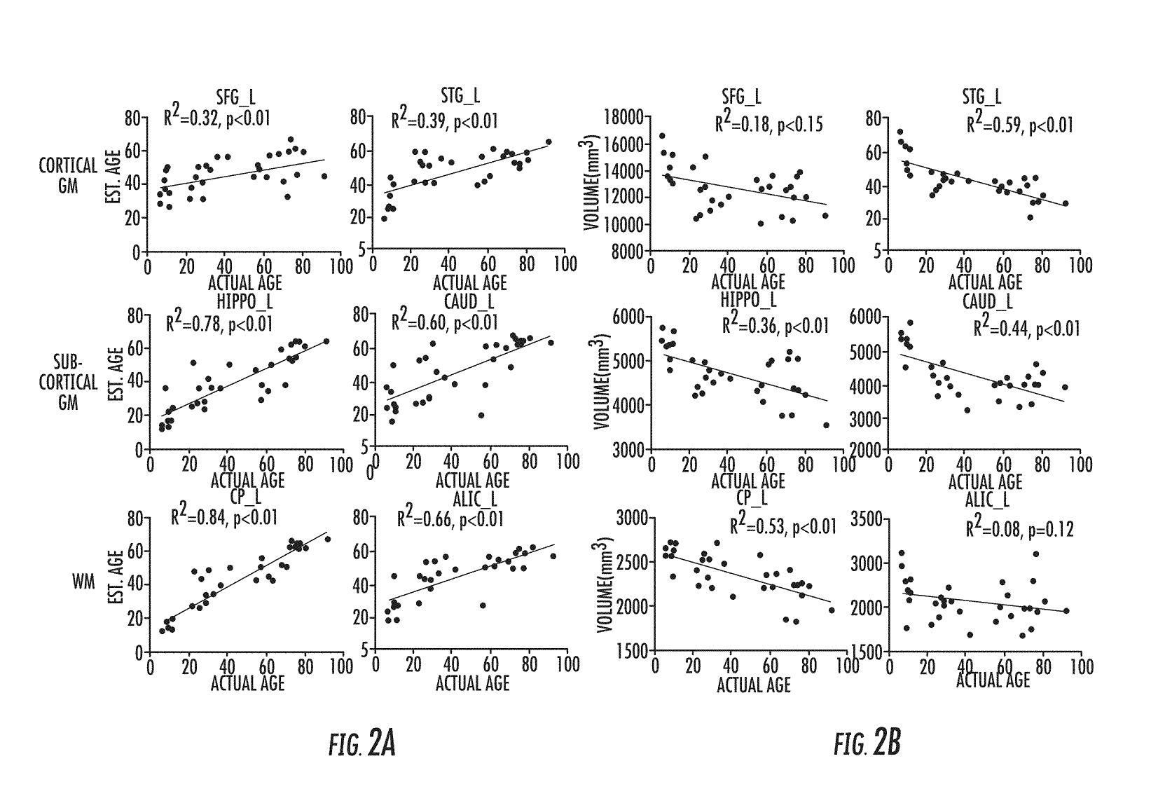

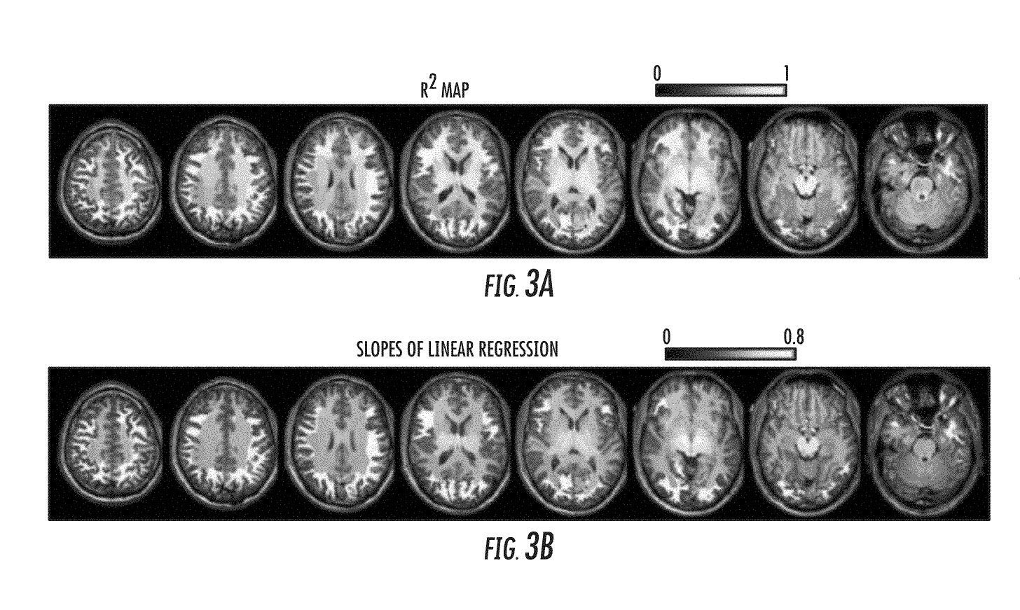

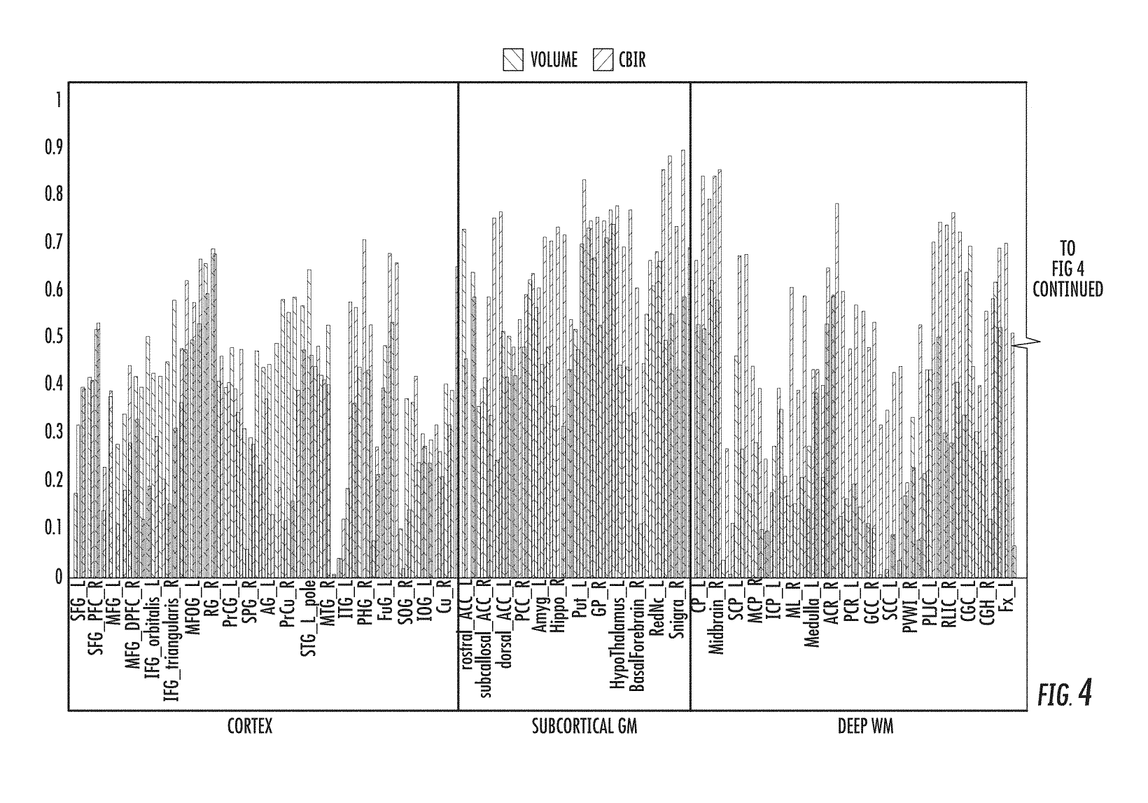

Testing of location-based feature extraction using age: One interesting test that can be performed with CBIR is the estimation of age. Because the exact age of each subject was known, the accuracy of the CBIR approach for age estimation could be evaluated. The aging probability was estimated in each test subject on a structure-by-structure basis according to Equation 4. The linear regression between the estimated ages and actual ages showed significant correlations in many structures. FIGS. 2A and 2B show the correlations in several structures in the cortical, subcortical gray matter, and white matter regions. The subcortical structures and deep white matter structures demonstrated high correlation between the estimated age (y-axes) and actual age (x-axes), with R² values around 0.7. The correlation in cortical structures was relatively weak, with R² around 0.3-0.5. The R² values and the slopes of linear regression were mapped to the T1-weighted images, and masked by a familywise p-value threshold of 0.05 (FIGS. 3A and 3B). The R² maps indicated that the age estimation is most precise in the subcortical gray matter, the anterior deep white matter, and the cerebellum. Some peripheral white matter tracts and gyri in the posterior and superior brain did not show significant correlation. The correlation coefficients, which represent the systematic bias between the estimated and actual ages, suggested high accuracy in the thalamus and midbrain structures and a higher degree of bias in the peripheral structure.

FIGS. 2A and 2B illustrate graphical views of data according to an embodiment of the present invention. FIG. 2A illustrates a linear regression between the estimated ages (y-axes) and actual ages (x-axes) of 30 test subjects in several cortical, subcortical gray matter, and white matter structures. FIG. 2B illustrates a linear regression between the structural volumes (y-axes) and ages (x-axes) in the same structures as in FIG. 2A. The R² and p values of the linear regression are denoted in each graph. Abbreviations: SFG_L-left superior frontal gyrus; STG_L-left superior temporal gyms; Hippo_L-left hippocampus; Caud_L-left caudate; CP_L: left cerebral peduncle; ALIC_L-left anterior limb of the internal capsule.

FIGS. 3A and 3B illustrate image views of whole-brain mapping of the R² and linear correlation coefficients of the linear regression between the estimated age and actual age in each structure, overlaid on a T1-weighted image. Only structures with significant linear regression (family-wise p-value<0.05) are shown. Dark red indicates low R2 or correlation coefficients, and bright color indicates high values.

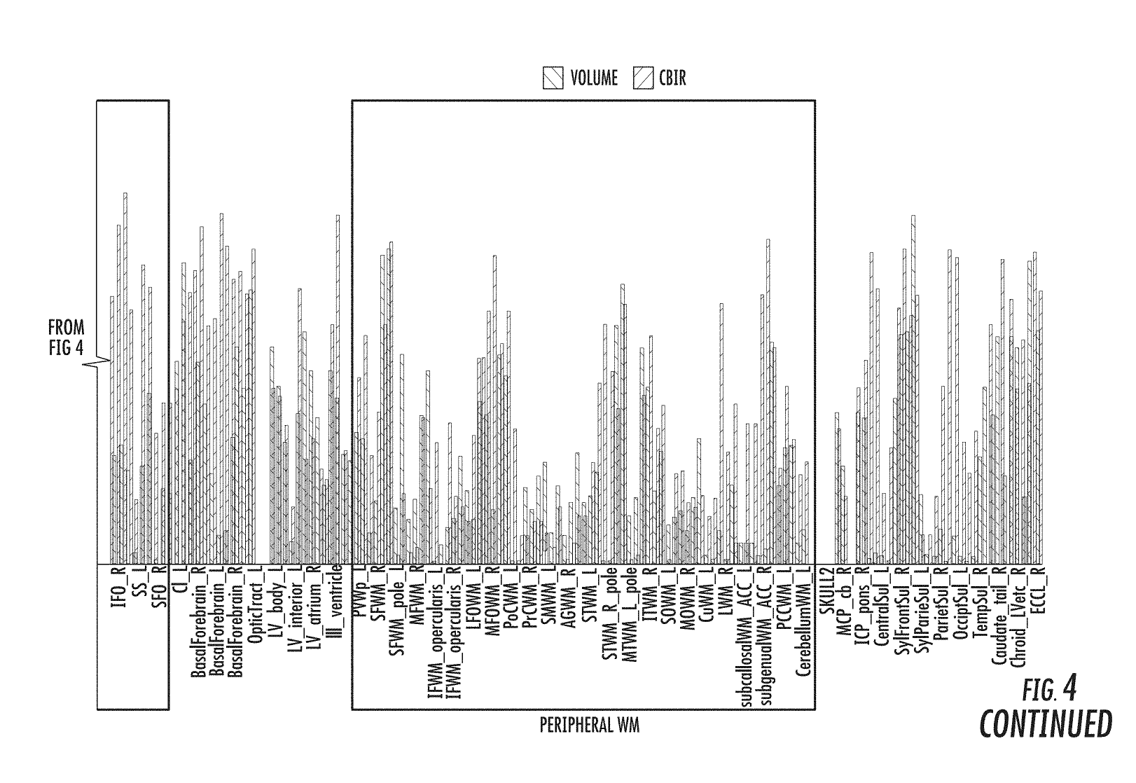

This CBIR-based age estimation was compared with a simple volume-based approach. Plots of the volume-to-age correlations are shown in FIG. 2B, in comparison to FIG. 2A. The R² values of volume-based and CBIR-based linear regression were directly compared in all 289 structures in FIG. 4. FIG. 4 illustrates graphical views of R² of the linear regression between the structural volume and age (dark grey bar), compared to the R² of the linear regression between the CBIR-based estimation and age, in 289 structures over the whole brain. In the subcortical gray matter and deep white matter, the CBIR-based age estimation outperformed volume-based estimation; whereas in the cortical structures, the R² of volume-based correlation was relatively higher. Overall, the highest accuracy levels were achieved by the CBIR-based approach in the subcortical gray matter, deep white matter, and several ventricle structures, reaching an R² of nearly 0.8 or higher. With an arbitrary threshold at R²>0.7, 48 structures reached this accuracy level with the CBIR-based approach, while there were only six structures that met this criteria with the volume-based approach.

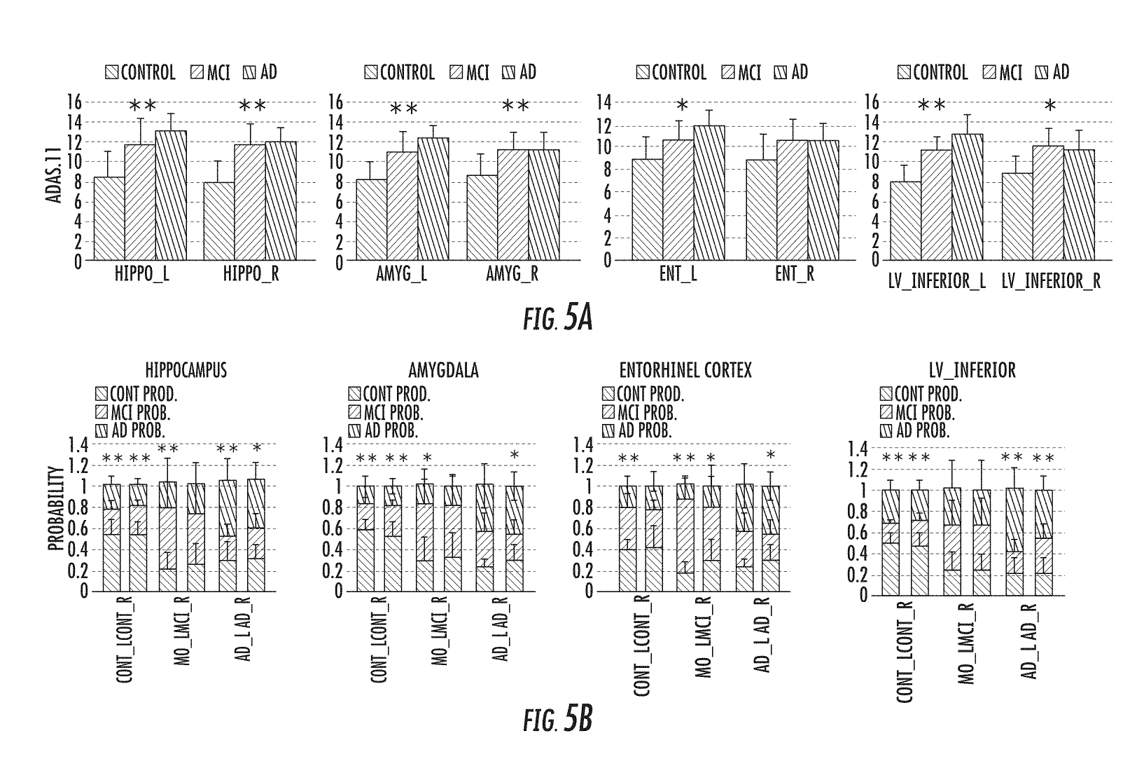

The cognitive assessment was estimated (ADAS.11 score) for the ADNI subjects using the disease-specific, multi-atlas pool according to Equation 4. The group average ADAS.11 estimated in the normal elderly was reviewed, MCI, and AD test subjects (n=10 each). Several structures of interest, such as the bilateral hippocampus and inferior lateral ventricle, the left amygdala, and the left entorhinal cortex, showed significantly different ADAS.11 estimation between test groups, based on one-way ANOVA (FIG. 5A). The disease group probability was also estimated (FIG. 5B) in these structures, estimated based on Equation 5. It was clear that the control subjects had higher control probabilities (more similar to controls), and MCI/AD subjects had higher MCI/AD probabilities, respectively. The differences between the control/MCI/AD probabilities in each test group were denoted by *p<0.05 and **p<0.005 using ANOVA.

FIGS. 5A and 5B illustrate graphical views of dementia probabilities and control/MCI/AD probabilities. FIG. 5A illustrates CBIR-based estimation of ADAS.11 scores in the control, MCI, and AD test subjects in the left and right hippocampus, amygdala, entorhinal cortex, and inferior lateral ventricle. The data are presented as group mean±standard deviation (n=10 in each group). * denotes a p-value<0.05, and ** denotes a p-value<0.01 by one-way ANOVA test between the groups, followed by FDR correction. FIG. 5B illustrates CBIR-based estimation of control probabilities (medium grey bars), MCI probabilities (light grey bars), and AD probabilities (dark grey bars) in the control, MCI, and AD test subjects in the same structures as in FIG. 5A. The labels under the stacked bars denote the subject groups and the left/right sides of the structures, for example, “Cont_L” in the first panel “Hippocampus” denotes left hippocampus in the controls. * denotes a p-value<0.05, and ** denotes a p-value<0.01 by one way ANOVA between the three probabilities in each subject group.

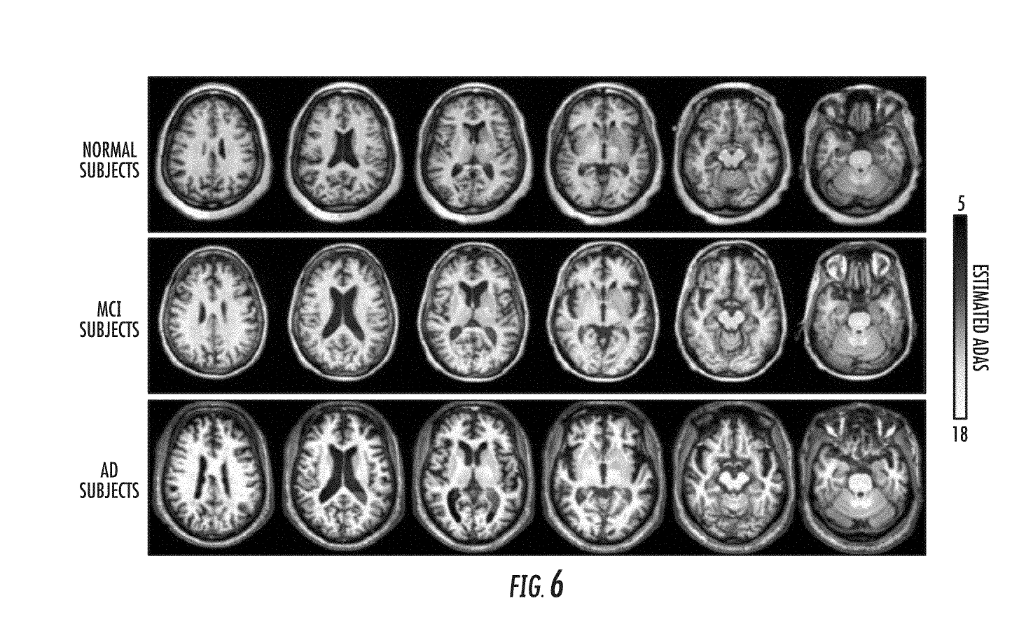

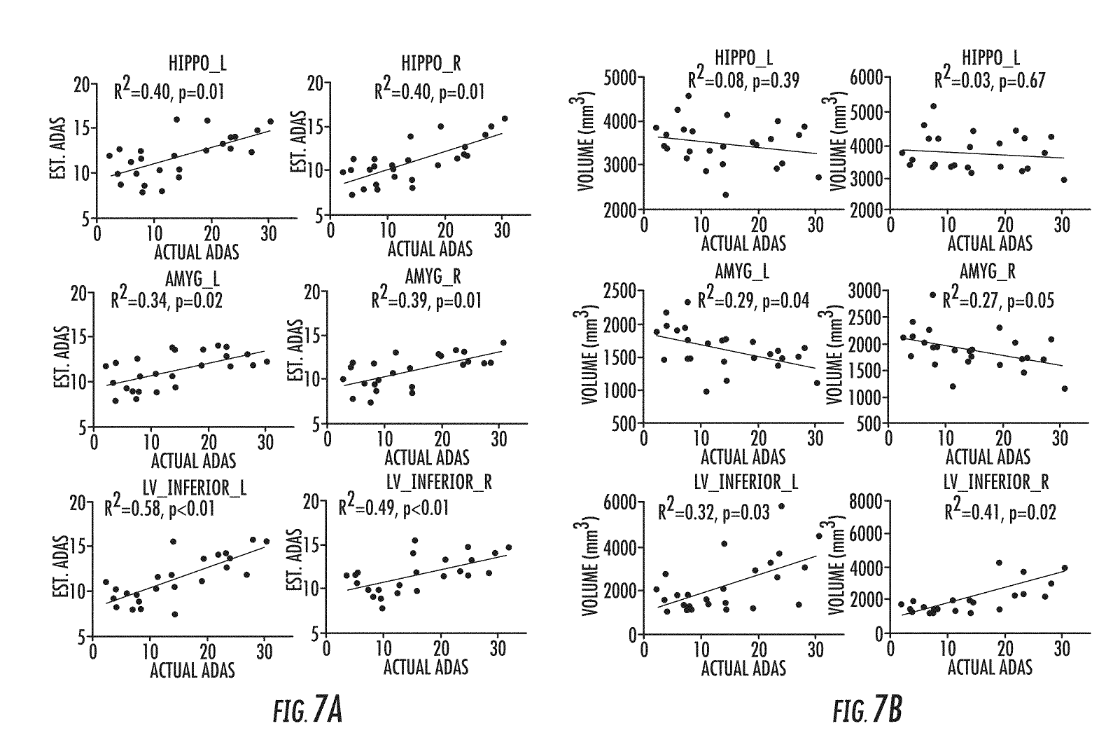

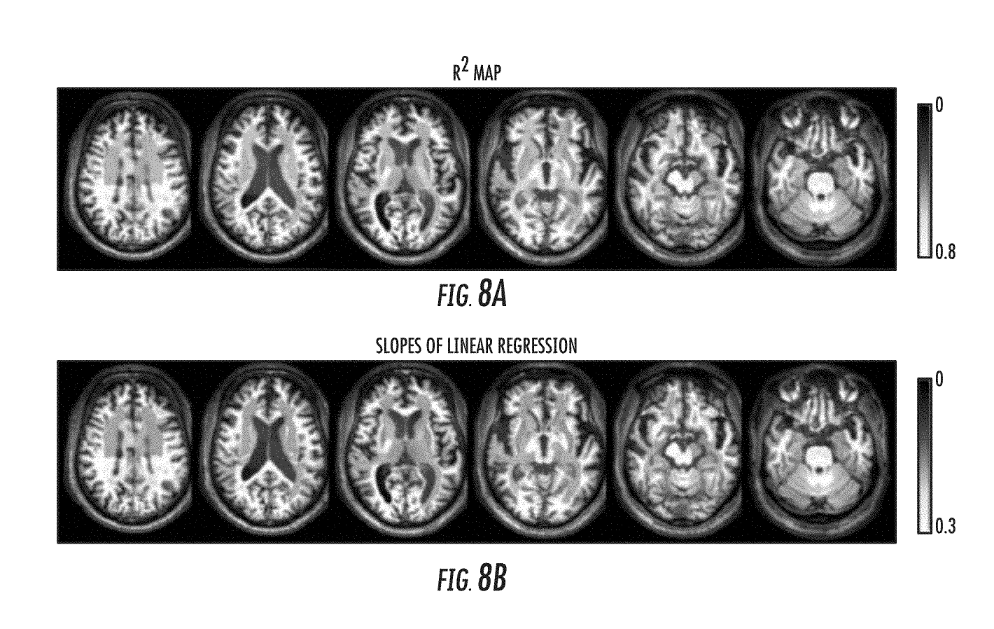

FIG. 6 illustrates whole-brain mapping of the estimated ADAS.11 scores in the normal elderly, MCI, and AD test subjects. The overlaid color map indicates the group mean, and only structures with significant group differences (family-wise p-value<0.05 by ANOVA test) are mapped. FIG. 6 shows the whole-brain mapping of the average ADAS.11 estimated in three test groups, and only the structures with significant group differences with a family-wise p-value<0.05 from ANOVA were color-coded. The ADAS.11 scores were significantly lower in normal elderly (dark red) and higher in AD subjects (bright), as well as highly lateralized in the left brain, such as the left amygdala, the caudate, the putamen, the globus pallidus, the entorhinal gyrus, the parahippocampal gyrus, and parts of the periventricular white matter and internal capsule. Linear correlation between the estimated ADAS.11 (y-axes) and actual scores (x-axes) are plotted in FIG. 7A for several key structures. In these plots, unlike in FIG. 5A, the data from all NC/MCI/AD groups were plotted without binning the data in each diagnostic category. The hippocampus and inferior lateral ventricle that surrounds the hippocampus showed relatively high correlation (R²=0.4-0.6), followed by the amygdala (R²=0.3-0.4). In comparison, the correlations between volumes and ADAS.11 are much lower in these key structures (FIG. 7A). The R² maps (FIG. 8A) showed high correlations in the hippocampus, the amygdala, the caudate, the thalamus, the internal capsule, the corona radiata, and the lateral ventricle, among others, with lateralization in some structures. The slopes of the linear regression (FIG. 8B) were highest in the hippocampus and inferior lateral ventricle.

FIGS. 7A and 7B illustrate graphical views of linear regressions, according to an embodiment of the present invention. FIG. 7A illustrates a linear regression between the estimated ADAS.11 score (y-axes) and actual score (x-axes) of 30 test subjects in the left and right hippocampus, amygdala, and inferior lateral ventricle. FIG. 7B illustrates a linear regression between the structural volumes (y-axes) and ADAS.11 score (x-axes) in the same structures. The R² and p values of the linear regression are denoted in each graph.

FIGS. 8A and 8B illustrate image views of whole brain mapping of the R2, according to an embodiment of the present invention. FIG. 8A illustrates linear correlation coefficients and FIG. 8B illustrates the linear regression between the estimated ADAS.11 and actual score in each structure, overlaid on a T1-weighted image. Only structures with significant linear regression (family-wise p-value<0.05) are shown.

MRI atlases are commonly used for automated image segmentation, which provide pre-segmented maps as a priori knowledge about the shapes and locations of the structures to guide the segmentation. The use of multiple atlases yields robust and accurate segmentation, as the rich anatomical information from multiple atlases offers the flexibility to accommodate the diverse anatomy of the patient population. The end-goal of the atlas- or multi-atlas-based approach is typically to obtain accurate segmentation, from which information about volumes, intensities, or shapes of the segmented structures can be extracted and compared among populations. Much of the previous effort has been focused on improving the segmentation accuracy through advanced image registration. The determination of the structural volumes is usually NOT the ultimate goal. Instead, the volume information is used to, for example, differentiate populations (and thus, can serve as a biomarker for diagnosis) or correlate the brain function measures (and thus, can predict the functional outcomes). Therefore, the volume information is an intermediate marker to extract more clinically meaningful information about the patients, such as diagnosis, prognosis, and functional risk factors.

During the multi-atlas segmentation, demographic and clinical information from the atlases is not available or is unused once satisfactory segmentation accuracy is achieved. The present invention is directed to a CBIR-based approach that enabled retrieval of such information from the atlas database and use it to estimate the unknown attributes of new patients. In other words, each atlas is considered a classifier, and the opinion from multiple classifiers are rated and fused to reach the final decision. In this respect, the meaning of the multi atlas library changes. If one is merely interested in segmentation accuracy, a question like, “what is the minimum number of atlases that would be required to achieve accurate segmentation?” is meaningful, but if the multi-atlas library is considered a knowledge database from which patient attributes are extracted, it needs to be enriched by cases with various anatomical and pathological conditions, as well as comprehensive demographic and clinical information. The present invention is directed to use of the multi-atlas analysis within the context of CBIR.

This CBIR-based disease estimation system is naturally incorporated in the multi-atlas selection processes. Without other prior information, it is assumed that the images with similar anatomical features share similar demographics and diagnostics. Searching for proper atlases among the multi-atlas pool relies on a similarity measure that weights the contribution of each atlas in decision-making. Intensity-based atlas-weighting is widely used as a similarity criterion, e.g., the intensity differences, cross-correlation, or mutual information. Shaped-based averaging is also an option, which requires initialization of labeling in the target image. The deformation energy of transformation between the atlases and targets can also be used, as less deformation indicates higher similarity between the images in the native space. The atlas weighting can be evaluated on a global scale, such as the whole brain, or localized scales, such as the voxels and structures. Defining weights locally improved the segmentation compared to global approach.

For diagnostic purposes, the atlas-weighting was computed on a structure-by-structure basis to reflect the local pathology and to best match the radiologists' reading convention. Compared to the voxel-by-voxel approach, weighting of an entire structure, which includes thousands of voxels, may not be sensitive to local mismatches. The strategy of the present invention is to focus on the boundary voxels of each structural label, assuming that the intensity of voxels inside the boundary is relatively homogeneous and registration mismatch is mostly reflected on the boundaries. Furthermore, the boundary of a structure is influenced by the shape of the structure of interest and by the surrounding anatomical features. For example, the medial, lateral, and dorsal surfaces of the hippocampus are surrounded by the ventricles. In many brains, the large portions of the ventricles in the dorsal and lateral surfaces are closed (invisible on MRI with 1 mm resolution) and the adjacent white matter tissues seem attached to the hippocampus, while these ventricle spaces enlarge and become visible in patients with severe brain atrophy.

The atlas-weighting scheme of the present invention is based on intensity-matching at the structural boundaries, and thus, the atlases that share not only similar hippocampal shapes, but also the surrounding ventricle anatomy, would receive higher weighting. This concept worked better and led to higher age-estimation accuracy for the subcortical gray matter and deep white matter structures that tend to have simpler anatomical boundaries; but it did not perform as well for the cortical gyri, where it is extremely difficult to achieve accurate boundary-to-boundary registration between atlases and targets. The deformation cost is also incorporated in the atlas-weighting, because the registration process itself is an effort to maximize the similarity between the atlases and targets. In order to obtain the atlas-target similarity in their native space for diagnostic purposes, both the deformation energy and the image similarity after deformation were taken into account.

The results of using the present invention demonstrate the feasibility of CBIR-based demographic and diagnosis estimation. The age estimation tested in this testing of the present invention may not have high clinical importance, but as the exact age was known, it was an ideal model with which to test the accuracy of the approach. The majority of the brain structures showed high correlation between the estimated and actual ages, especially in the subcortical gray matter and the deep white matter (R²=0.7-0.8). However, it should be noted that there was an overestimation of age in the pediatric population and an underestimation for the elderly population, leading to a regression slope of less than 1. This was likely due to the fact that the inaccuracy of age estimations of these two boundary populations led to inclusion of older atlases for age estimation of the pediatric population and younger atlases for the elderly population in the weighting process. This bias, however, can potentially be corrected based on the training data. The spatial difference in the R² maps and correlation slopes showed the estimation accuracy and precision varied from structure to structure, which in turn, indicated that the sensitivity of the atlas-weighting and the degree of age-dependent anatomical difference varies from structure to structure. FIGS. 3A and 3B indicate that the combination of the CBIR- and volume-based analysis could be a viable option to maximize the efficacy of feature-extraction.

CBIR-based diagnosis in the dementia population demonstrated significant differences between the normal elderly/MCI/AD groups in several key structures, such as the hippocampus, the amygdala, the entorhinal cortex, and the lateral ventricle, and the statistical power in these structures was higher than conventional volumetric measurements. The estimated ADAS.11 and the actual score agreed well in several structures in the subcortical gray matter, the deep white matter, and the ventricles. The correlation curves also showed overestimation and underestimation on the lower and upper ends of the ADAS.11 spectrum, respectively. The reason could be similar as explained above in the case of the age estimation. Note that the ADAS.11 or other cognitive assessments are coarse measures of Alzheimer's disease; whereas in the age test, the age is an exact measure. Because a diagnosis without pathological examination cannot be exact for AD, the diagnosis of the atlas data contains a certain degree of ambiguity, and thus, the estimated diagnosis is not expected to achieve perfect accuracy in reality. However, it is encouraging that the CBIR-based approach achieved significantly better accuracy than the conventional volume-based analysis.

The type of patient attributes estimated by the framework of the present invention can be extended to include imaging reports (from PET, CT, etc.) and non-imaging tests (neurocognitive tests, longitudinal functional changes, etc.). Finally, this approach would only be possible when rich multi-atlas repositories are available with the different types of pathology and associated diagnostic information.

The present invention carried out using a computer, non-transitory computer readable medium, or alternately a computing device or non-transitory computer readable medium incorporated into the scanner. Indeed, any suitable method of calculation known to or conceivable by one of skill in the art could be used. It should also be noted that while specific equations are detailed herein, variations on these equations can also be derived, and this application includes any such equation known to or conceivable by one of skill in the art.

A non-transitory computer readable medium is understood to mean any article of manufacture that can be read by a computer. Such non-transitory computer readable media includes, but is not limited to, magnetic media, such as a floppy disk, flexible disk, hard disk, reel-to-reel tape, cartridge tape, cassette tape or cards, optical media such as CD-ROM, writable compact disc, magneto-optical media in disc, tape or card form, and paper media, such as punched cards and paper tape.

The computing device can be a special computer designed specifically for this purpose. The computing device can be unique to the present invention and designed specifically to carry out the method of the present invention. Scanners generally have a console which is a proprietary master control center of the scanner designed specifically to carry out the operations of the scanner and receive the imaging data created by the scanner. Typically, this console is made up of a specialized computer, custom keyboard, and multiple monitors. There can be two different types of control consoles, one used by the scanner operator and the other used by the physician. The operator's console controls such variables as the thickness of the image, the amount of tube current/voltage, mechanical movement of the patient table and other radiographic technique factors. The physician's viewing console allows viewing of the images without interfering with the normal scanner operation. This console is capable of rudimentary image analysis. The operating console computer is a non-generic computer specifically designed by the scanner manufacturer for bilateral (input output) communication with the scanner. It is not a standard business or personal computer that can be purchased at a local store. Additionally this console computer carries out communications with the scanner through the execution of proprietary custom built software that is designed and written by the scanner manufacturer for the computer hardware to specifically operate the scanner hardware.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention. While exemplary embodiments are provided herein, these examples are not meant to be considered limiting. The examples are provided merely as a way to illustrate the present invention. Any suitable implementation of the present invention known to or conceivable by one of skill in the art could also be used.

What is claimed is:

1. A method for estimation of patient attributes comprising:
providing a database framework of multiple brain atlases;
weighing demographic and diagnostic information of the multiple brain atlases;
fusing the demographic and diagnostic information based on the weighing of the multiple brain atlases; and
generating an estimated diagnosis on a structure by structure basis.

2. The method of claim 1 further comprising using a database framework based on magnetic resonance (MR) images.

3. The method of claim 1 further comprising using a context-based image retrieval system.

4. The method of claim 1 further comprising estimating various types of disease and pathology with the choice of patient attributes.

5. The method of claim 1 further comprising diagnostic estimation in Alzheimer's disease.

6. The method of claim 1 further comprising building the multiple brain atlases with images from healthy volunteers with a wide range of age and pathological states.

7. The method of claim 1 further comprising performing multiple-atlas segmentation based on label-by-label atlas weighting.

8. The method of claim 1 further comprising using atlases containing a number of anatomical structures, wherein each structure has associated information for age, diagnosis, and interesting atlas properties.

9. The method of claim 8 further comprising building aging and diagnosis probability maps for each of the number of anatomical structures.

10. The method of claim 9 further comprising generating and displaying maps associated with the number of anatomical structures.

11. The method of claim 1 further comprising generating and displaying maps and visual representations of data associated with method.

12. A system for estimation of patient attributes comprising:
a database framework of multiple brain atlases; and
a non-transitory computer readable medium programmed for,
weighing demographic and diagnostic information of the multiple brain atlases;
fusing the demographic and diagnostic information based on the weighing of the multiple brain atlases; and
generating an estimated diagnosis on a structure by structure basis.

13. The system of claim 12 further comprising using a database framework based on magnetic resonance (MR) images.

14. The system of claim 12 further comprising using a context-based image retrieval system.

15. The system of claim 12 further comprising diagnostic estimation in Alzheimer's disease.

16. The system of claim 12 further comprising performing multiple-atlas segmentation based on label-by-label atlas weighting.

17. The system of claim 12 further comprising using atlases containing a number of anatomical structures, wherein each structure has associated information for age, diagnosis, and interesting atlas properties.

18. The system of claim 17 further comprising building aging and diagnosis probability maps for each of the number of anatomical structures.

19. The system of claim 18 further comprising generating and displaying maps associated with the number of anatomical structures.

20. The system of claim 12 further comprising generating and displaying maps and visual representations of data associated with method.
